(19) United States
(12) Patent Application Publication
(10) Pub. No.: US 2013/0247644 A1
(43) Pub. Date: Sep. 26, 2013

(54) IMPLANTABLE PRESSURE SENSOR

(71) Applicants: Marek Swoboda, Philadelphia, PA (US); Matias Gabriel Hochman, Philadelphia, PA (US); Mark Mattiucci, Philadelphia, PA (US); Fred Fritz, Philadelphia, PA (US)

(72) Inventors: Marek Swoboda, Philadelphia, PA (US); Matias Gabriel Hochman, Philadelphia, PA (US); Mark Mattiucci, Philadelphia, PA (US); Fred Fritz, Philadelphia, PA (US)

(73) Assignee: ICPCHECK INC., Bensalem, PA (US)

(21) Appl. No.: 13/990,185

(22) PCT Filed: Dec. 8, 2011
(86) PCT No.: PCT/US2011/063935

§ 371 Date: May 29, 2013

Related U.S. Application Data
(60) Provisional application No. 61/459,229, filed on Dec. 10, 2010.

(51) Int. Cl. G01L 25/00; A61B 5/00; A61B 5/03

(52) U.S. Cl. CPC G01L 25/00; A61B 5/031; A61B 5/686
USPC 73/1.08; 600/561

(57) ABSTRACT

A pressure sensor that is implantable within a living being that wirelessly provides pressure data within the living being to a wireless receiver. The pressure sensor includes an elastic membrane to which at least one capacitive actuator is coupled for applying a known force to the membrane to determine membrane characteristics. The pressure sensor includes a force transducer contacting the membrane for determining the pressure within the living being and which includes an internal calibrating force mechanism. This calibrating force mechanism permits force transducer displacement away from the membrane where a zero force transducer reading is taken and then applying a calibrating force and taking another reading. From these two points, a force transducer characteristic is derived and, along with membrane characteristics, an accurate pressure within the living being is obtained from the sensor. An alternative embodiment replaces the capacitive actuators with a known mass and an external vibratory source.

### CROSS-REFERENCE TO RELATED APPLICATIONS

This PCT application claims the benefit under 35 U.S.C. §119(e) of Provisional Application Ser. No. 61/459,229 filed on Dec. 10, 2010 entitled IMPLANTABLE PRESSURE SENSOR and all of whose entire disclosure is incorporated by reference herein.

### BACKGROUND OF THE INVENTION

1. Field of Invention

This present invention generally relates to medical devices and more particularly to implantable devices for monitoring internal pressure, e.g., intracranial pressure, of a living being.

2. Description of Related Art

Implantable sensors are important diagnostic devices which help measure physiological parameters that are difficult or even impossible to measure noninvasively. However, implantable devices pose several problems for the designer. They have to be biocompatible, so they do not harm the patient over a long or short term, and they cannot trigger physiological or patho-physiological reactions (e.g., immunological reactions) which can compromise their ability to perform measurements.

Another set of problems stems from engineering requirements. The stability requirements for the implantable sensor are more strict that those for the noninvasive devices since they cannot be calibrated at will, or at least, the calibration process is usually more challenging compared to other devices.

The long term implantable pressure sensors carry two inherent problems affecting their stability.

First, short term body temperature fluctuations change the internal temperature, thus changing the internal pressure. This pressure change affects the pressure differential between the internal pressure of the device and the external one (e.g., intracranial pressure, ICP). Another short term factor may include the change in the amount of gas inside the sensor body (e.g., gas absorption due to oxidation or gas release from materials inside the capsule). These types of changes can also add or subtract from forces acting on the transducer by changing forces acting on the membrane separating the inside of the sensor from the external environment.

Second, the natural body responses cause protein deposits on the outside surface of the device, thereby changing the effective stiffness of the membrane. This change in effective stiffness may change the sensitivity of the device or even entirely block the external pressure. This type of problem is usually associated with long term changes.

The above-listed problems (assuming that the membrane by itself does not generate any stress on the sensor regardless of the displacement, i.e., an ideal membrane) causes the output-input characteristic of the sensor to shift up or down (see FIG. 7A); or to rotate about certain point changing the slope of the characteristic (FIG. 7B). In particular, plot 51 of FIG. 7A depicts the undisturbed input-output characteristic. Plot 52 depicts the input-output characteristic of the internal pressure (i.e., inside the sensor body) which is lowered. Plot 53 depicts the input-output characteristic if the internal pressure is elevated.

One of the physiological parameters which is difficult to measure noninvasively is ICP. ICP can be an important parameter in monitoring hydrocephalic patients, or traumatic brain injury (TBI) victims.

Since cerebrospinal fluid is enclosed in a semi closed system (i.e., the skull), the forces exerted by it are counterbalanced by a rigid structure of bones and, to some extent, by a semi rigid structure of the spinal channel. In a mechanical sense, there is no direct link (except for some small vessels which are difficult to utilize due to their anatomical nature) between the cerebrospinal fluid and the external environment. Thus, an implantable sensor outfitted with a reliable means of calibration would be a valuable addition to neurosurgical armamentarium.

Thus, there remains a need for an implantable pressure sensor that can account for these artifacts and provide a more accurate reading of the internal pressure to be measured.

All references cited herein are incorporated herein by reference in their entireties.

### BRIEF SUMMARY OF THE INVENTION

A pressure sensor that is implantable within a living being for detecting a pressure (e.g., intracranial pressure (ICP), blood pressure, lung pressure, etc.) present at a location wherein the pressure sensor is implanted is disclosed. The implantable pressure sensor comprises: a housing comprising one side formed by a flexible membrane; wherein the housing further comprises sensor electronics including a force transducer which is in contact with the membrane for detecting flexing of the flexible membrane when the flexible membrane is exposed to the pressure present at the location; the sensor electronics further comprise at least one capacitor coupled to the flexible membrane, wherein the at least one capacitor applies a known force to the membrane, detected by the force transducer, when the at least one capacitor is energized by the sensor electronics; and wherein the known force is used to calibrate for a stiffness associated with the flexible membrane in measuring the pressure at the location.

A pressure sensor that is implantable within a living being for detecting a pressure (e.g., intracranial pressure (ICP), blood pressure, lung pressure, etc.) present at a location wherein the pressure sensor is implanted is disclosed. The implantable pressure sensor comprises: a housing comprising one side formed by a flexible membrane; the housing further comprises sensor electronics including a displaceable force transducer in contact with the membrane for detecting flexing of the flexible membrane when the flexible membrane is exposed to the pressure present at the location; the sensor electronics further comprise a calibrating force member that applies a known calibrating force to the force transducer when the force transducer is displaced away from the flexible membrane; and wherein the known force is used, along with a zero pressure value obtained when the force transducer is displaced away from the membrane and without application of the known calibrating force, to form a force transducer characteristic which regulates all future force transducer measurements.

A method for calibrating a pressure sensor in situ within a living being for detecting a pressure (e.g., intracranial pressure (ICP), blood pressure, lung pressure, etc.) present at a location within the living being is disclosed. The method comprises: disposing a pressure sensor within the living being wherein the pressure sensor comprises a force transducer in contact with a flexible membrane, forming a portion of an outer surface of said pressure sensor, that is exposed to the pressure present at the location; coupling a capacitor to the flexible membrane; energizing the capacitor with a plurality of energy levels to apply corresponding known forces to the flexible membrane; and collecting the force transducer outputs corresponding to the applied known forces to generate a flexible membrane characteristic that is used to account for membrane stiffness which regulates all future force transducer measurements.

A method for calibrating a pressure sensor in situ within a living being for detecting a pressure (e.g., intracranial pressure (ICP), blood pressure, lung pressure, etc.) present at a location within the living being is disclosed. The method comprises: disposing a pressure sensor within the living being wherein the pressure sensor comprises a force transducer in contact with a flexible membrane, forming a portion of an outer surface of said pressure sensor, that is exposed to the pressure present at the location; displacing the force transducer away from the flexible membrane; collecting a force transducer output with the force transducer displaced out of contact with the flexible membrane to obtain a zero pressure value; applying at least one known calibrating force to the force transducer and collecting a corresponding force transducer output; and generating a force transducer characteristic from the zero pressure value and the corresponding force transducer output which regulates all future force transducer measurements.

A pressure sensor that is implantable within a living being for detecting a pressure (e.g., intracranial pressure (ICP), blood pressure, lung pressure, etc.) present at a location wherein the pressure sensor is implanted is disclosed. The implantable pressure sensor comprises: a housing comprising one side formed by a flexible membrane; wherein the housing further comprises sensor electronics including a displaceable force transducer in contact with the membrane for detecting flexing of the flexible membrane when the flexible membrane is exposed to the pressure present at the location. The flexible member comprises a known mass coupled thereto; wherein the sensor electronics further comprise a processor coupled to at least one detector for detecting the displacement of the mass when a known vibratory force is applied to the flexible membrane; and wherein the processor calculates a calibration force based on the displacement of the mass and time of displacement of the mass to form a force transducer characteristic which regulates all future force transducer measurements.

A method for calibrating a pressure sensor in situ within a living being for detecting a pressure (e.g., intracranial pressure (ICP), blood pressure, lung pressure, etc.) present at a location within the living being is disclosed. The method comprises: disposing a pressure sensor within the living being wherein the pressure sensor comprises a force transducer in contact with a flexible membrane, forming a portion of an outer surface of the pressure sensor, that is exposed to the pressure present at the location and wherein a known mass is coupled to the flexible membrane; applying a known vibratory force to the flexible membrane and collecting displacement data of the known mass; and generating a force transducer characteristic from the displacement data which regulates all future force transducer measurements.

### BRIEF DESCRIPTION OF SEVERAL VIEWS OF THE DRAWINGS

The invention will be described in conjunction with the following drawings in which like reference numerals designate like elements and wherein:

FIG. 1 is an enlarged cross-sectional view of the implantable sensor of the present invention;

FIG. 2 is an enlarged cross-sectional view of the implantable sensor of the present invention including a transparent window for infrared communication;

FIG. 2A is a block diagram of the implantable sensor of FIG. 2;

FIG. 3 depicts how the implantable sensor is positioned within the living being, e.g., within the head of a human, and how the implantable sensor communicates with an external hand-held portion;

FIG. 4 is a partial view of the head of a living being wherein the implantable sensor is placed within the subarachnoid space and which allows for infrared or radio communication with the handheld device;

FIG. 5 depicts another preferred embodiment of the implantable sensor wherein the transducer-membrane assembly portion of the implantable sensor is placed at a distal end of a catheter and the transceiver portion of the sensor is positioned at a proximal end of the catheter for communicating with the hand-held device;

FIG. 6 is an enlarged view of the proximal end (A) and of the distal end (B) of the embodiment of FIG. 5;

FIG. 7A is a prior art graph that depicts how the input-output relationship changes with internal (i.e., inside the sensor body) pressure;

FIG. 7B is a prior art graph that shows how the input-output relationship changes due to protein buildup on the surface of the sensor;

FIG. 8A is a graph that shows an example of a three point calibration, where force F1, F2 and F3 are generated by an actuator (e.g., capacitive actuator) attached to the membrane and the sensor's body;

FIG. 8B is a graph of an ICP-output characteristic obtained from the force output characteristic of FIG. 8A;

FIG. 9 is a prior art graph showing how changes in temperature affect sensor sensitivity;

FIG. 9A is a functional diagram of the force transducer's sensing element comprising a sensitive membrane and a diaphragm, the former of which is in direct contact with the invention's membrane;

FIG. 10 is a flow diagram showing how the calibration of the implantable sensor of the present invention is achieved;

FIG. 11 is a partial cross sectional view of the force transducer and displacement actuator taken along line 11-11 of FIG. 2 which omits the calibrating force mechanism;

FIG. 12A is a view similar to FIG. 11 showing the force transducer in a displaced condition and showing the calibrating force mechanism in position to apply a calibrating force to the force transducer;

FIG. 12B is a view similar to FIG. 11 showing the force transducer in its operative position and showing the calibrating force mechanism displaced away from the force transducer;

FIG. 13A is a partial view of the implantable sensor that does not utilize a capacitive actuator but rather uses a vibratory calibration configuration;

FIG. 13B is similar to the device of FIG. 13A but with the force transducer displaced away from the membrane.

### DETAILED DESCRIPTION OF THE INVENTION

The invention of the present application thereof, it will be apparent to one skilled in the art that various changes and modifications can be made therein without departing from the spirit and scope thereof.

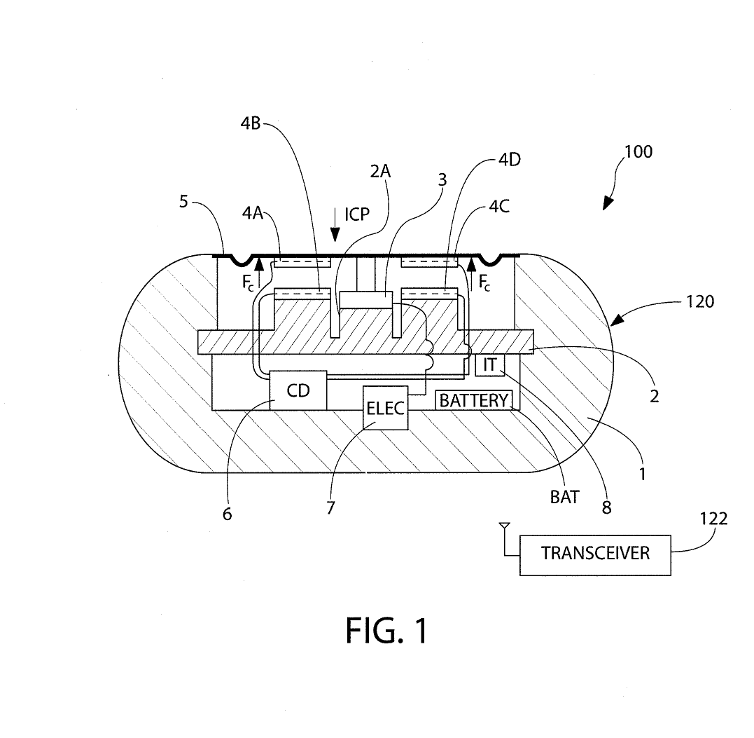

As shown in FIG. 1, the present invention 100 comprises an implantable pressure sensor 120 and a remotely-located transceiver 122. As a result, internal pressure data obtained from the implantable sensor 120 is then transmitted wirelessly to the remotely-located transceiver 122.

The implantable pressure sensor 120 comprises a rigid housing 1 having an elastic or flexible membrane 5 that houses an electronics board 2, with a force transducer 3 disposed between the board 2 and the membrane 3. The sensor 120 comprises at least one capacitor (4A/4B or 4C/4D), each of which has one capacitor plate (4A and 4C) coupled to an inside surface of the membrane 3. The corresponding capacitor plates (4B and 4D) are attached to a surface of the electronics board 2 in alignment with their respective pairing capacitor plates, 4A and 4C. As will be discussed in detail later, when energized, these capacitors (4A/4B, 4C/4D) generate a force F_{c} that can push or pull the membrane 3; as a result, these capacitors are termed “capacitive actuators”. The implantable sensor 120 further comprises a charging device (CD) 6 that charges/discharges the capacitors 4A/4B and 4C/4D. As mentioned previously, the sensor 120 includes a communication mechanism (IT) 8 for wirelessly transmitting collected pressure data to the transceiver 122. As will be discussed in detail later, the communication format may include radio communication, infrared communication, etc., and the present invention is not limited to any particular communication methodology. It should be noted that the term “capacitor plate” can also be referred to as “electrode”.

The sensor 120 also comprises a battery BAT for powering force transducer electronics (ELEC) 7 the charging device 6 and the communication device IT 8. The battery BAT may be a rechargeable type, receiving a recharge signal from the remotely-located transceiver 122. It should be understood that the battery BAT is by way of example only and that the implantable sensor 120 may be a passive device that receives its electrical energy from the remotely-located transceiver 122 or other well-known external recharge device.

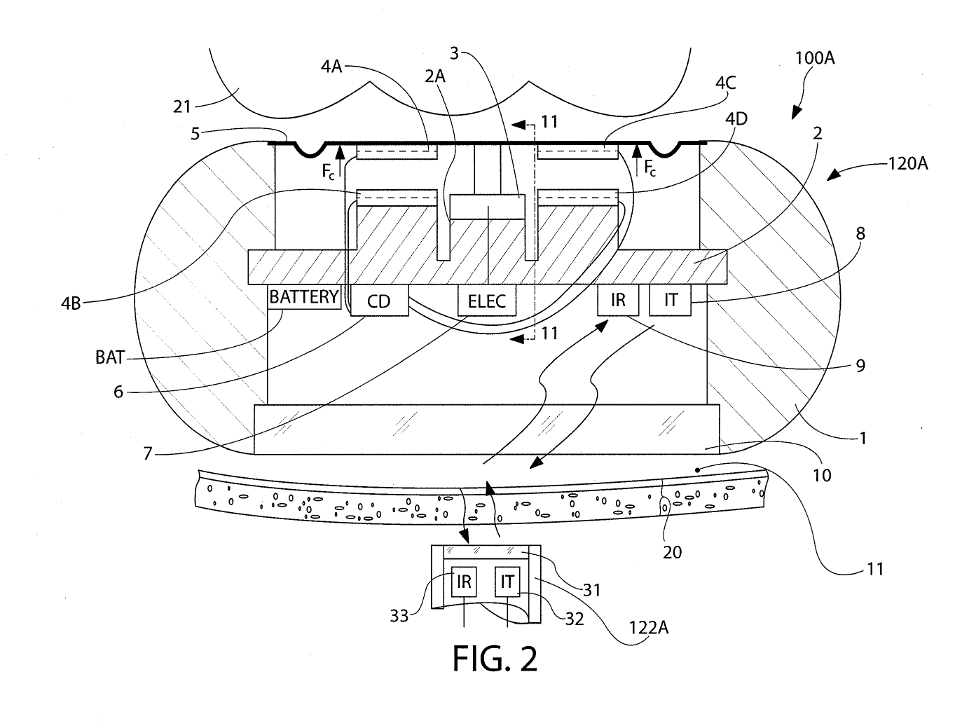

FIG. 2 discloses an alternative embodiment 100A to the first embodiment 100 in that the communication mechanism is an infrared communication mechanism. In particular, the implantable sensor 120A includes a communication mechanism having an LED transmitter 8 (e.g., emitter OP200 by TT Electronics) and an LED receiver 9 (phototransistor OP500 by TT Electronics). Thus, measured internal pressure values can be detected by the sensor 120A and then transmitted out of the living being to a remotely-located infrared transceiver 122A. Similarly, the LED receiver 9 can be used to receive electromagnetic energy (e.g., infrared light) to charge the battery BAT or, if the implantable sensor is a passive device, to charge the charge device for actuating the capacitive actuators.

To effect the infrared communication, the side of the sensor housing 1 directly opposite the transmitter 8/receiver 9 pair comprises a transparent material (e.g., plexiglass) 10 that permits the passage of the infrared energy between the implantable sensor 120A and the infrared receiver 122A. By way of example only, when the implantable sensor 120A is to measure intracranial pressure (ICP), the sensor 120A is implanted within the subarachnoid space 11 of the test subject, as shown in FIG. 2, outside the brain 21, the infrared energy passes through the scalp, skull, dura and arachnoid matter (the combination indicated by the reference number 20). The infrared receiver 122A also comprises an infrared transmitter 32/receiver 33 pair for communicating with the implantable sensor 120A and also includes a transparent distal end 31 for allowing passage of the infrared energy.

Again, as with the first embodiment 100, this embodiment 100A may comprise a battery that is rechargeable, or alternatively, this embodiment 100A may be a passive device, receiving all of its energy from the transceiver 122A.

FIG. 2A provides a block diagram of the second embodiment 120 wherein the transducer electronics 7 includes a microcontroller 123 (e.g., MSP430xG461x Mixed Signal Microcontroller by Texas Instruments) and an amplifier 125 (e.g., OPA735 by Texas Instruements). When the force transducer 3 (e.g., a piezoresistive pressure sensor (e.g., low pressure sensor SM5103 or SM5106 by Silicon Microstructures Inc.) detects the pressure, its electrical signal corresponding to the pressure is first amplified by the amplifier 125 and is digitized by the microcontroller 123 before being wirelessly transmitted (e.g., an ICP signal) to the transceiver 122A via the emitter LED 8. An LED receiver 33 then passes this to a microcontroller 131 for processing and ultimate display 133 or other output to the operator or user. An emitter LED 32 provides input/commands to the implantable pressure sensor 120A.

It should be noted that the microcontroller 123 controls the operation of the sensor 120/120A, including the charging device 6, the transducer electronics 7, the capacitive actuators, the emitter LED 8 and, as will be discussed later, the actuator 144 and calibrating force member 148. Thus, all of these components, including the battery BAT are termed “sensor electronics”.

As mentioned earlier, implantable pressure sensor 120/120A is powered from the internal battery BAT or from the receiver 122/122A utilizing electromagnetic waves (RF or IR) transmitted through the skin, tissue and/or bone. The measured quantity, e.g., pressure, is detected using an active sensor principle where the energy from the measured quantity is amplified by the amplifier 125. In the preferred embodiment, information about the measured signal is converted to a frequency coded message and, for example, optically (e.g., infrared) transmitted outside the body to the receiver (see FIGS. 2-6). In the preferred embodiment (FIGS. 1-2A) the sensor remains idle inside human body. When the transceiver 122A is activated by the user, the transceiver 122A sends an infrared pulse to the sensor 120A. This signal wakes up (also referred to as a “start command”) the microcontroller 123 which controls the entire process in order to minimize power consumption. In particular, the steps to measure the signal by the sensor 120A are:
1) The microcontroller 123 turns on the force transducer (e.g., piezoresistive die) and its amplification system 125;
2) Digitizing of the measured quantity (e.g., ICP) value;
3) Frequency modulating the measured (e.g., ICP) value;
4) Transmitting the frequency via infrared energy;
5) Implantable sensor goes to sleep.

One problem that this configuration encounters is the occasional occurrence of the output signal (i.e., the measured quantity signal 142) triggering the microcontroller 123 when the working wavelength of the “wake-up” signal 140 (e.g., transmitted infrared signal) and the measured quantity signal 142 (e.g., ICP signal) are the same. This problem is solved by two different methods. A first solution uses software whereby the microcontroller 123 overrides the wake up interruption signal 140 until the measured quantity signal 142 is sent; however this reduces the availability of ports in the microcontroller 123. A second solution is the use of two different wavelengths for signals 140 and 142 that do not interfere with one another. The latter solution is the preferred method since it takes advantage of some microcontroller inherent hardware benefits that prevents false triggering of the implantable sensor 120A.

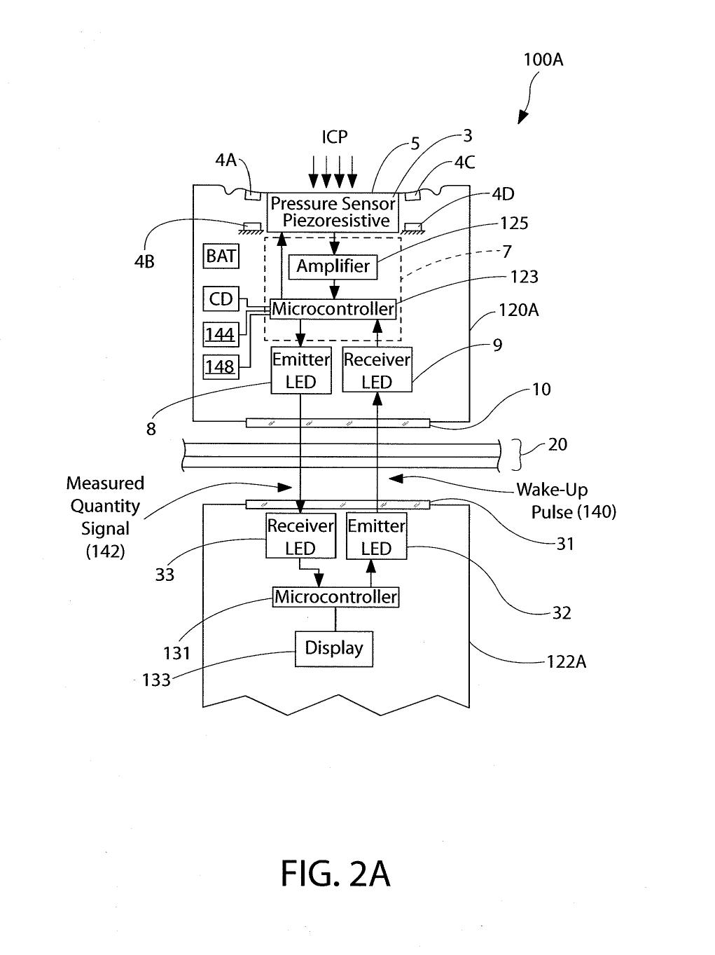

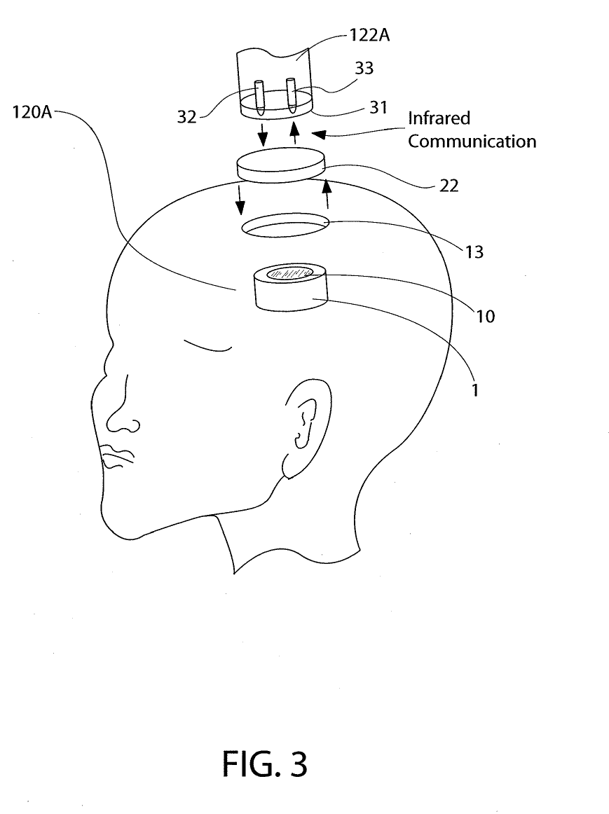

FIG. 3 depicts how the implantable sensor 120A is positioned when used to measure ICP. In particular, a piece 22 of the skull is removed during trepanation to form a burr hole 13 and permit implantation of the sensor 120A in brain, as discussed earlier with respect to FIG. 2. The sensor 120A is positioned with its transparent surface 10 facing outward to transmit/receive infrared energy outwardly of the skull towards the remotely-located transceiver 122A. Once the sensor 120A is positioned, the piece 22 of skull is re-inserted within the burr hole 13 and sensor 120A-transceiver 122A communication occurs as shown in FIG. 3. Therefore, although the implantable sensor 120A and the transceiver 122A require the use of respective transparent surfaces 10 and 31, infrared transmission through the scalp/skull/dura, arachnoid matter 20 does occur without major disruption of the infrared signals, as shown in FIG. 4.

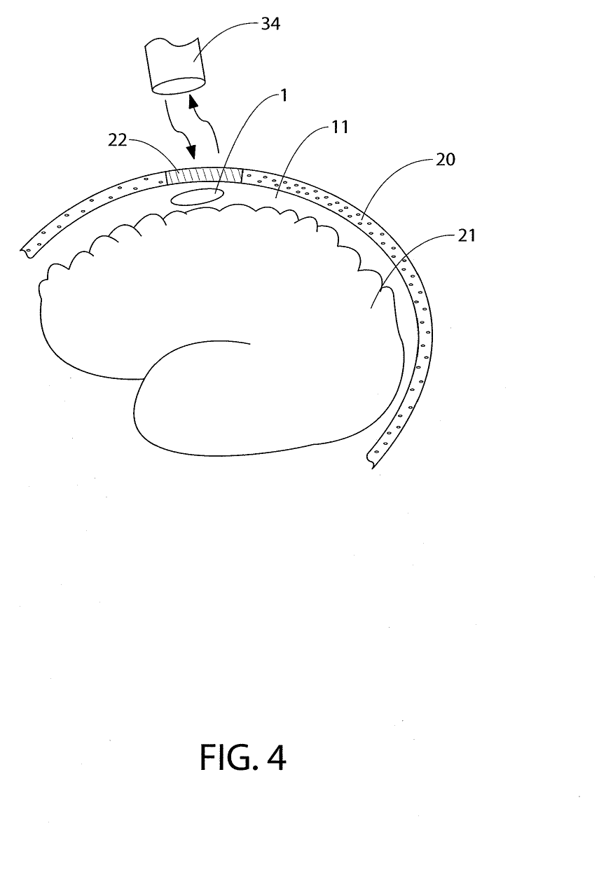

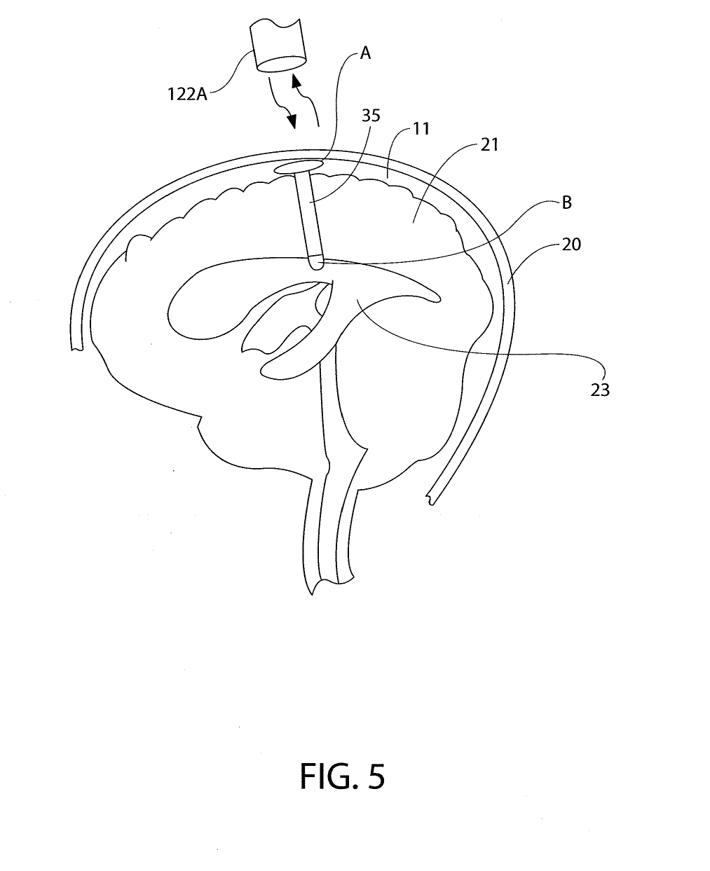

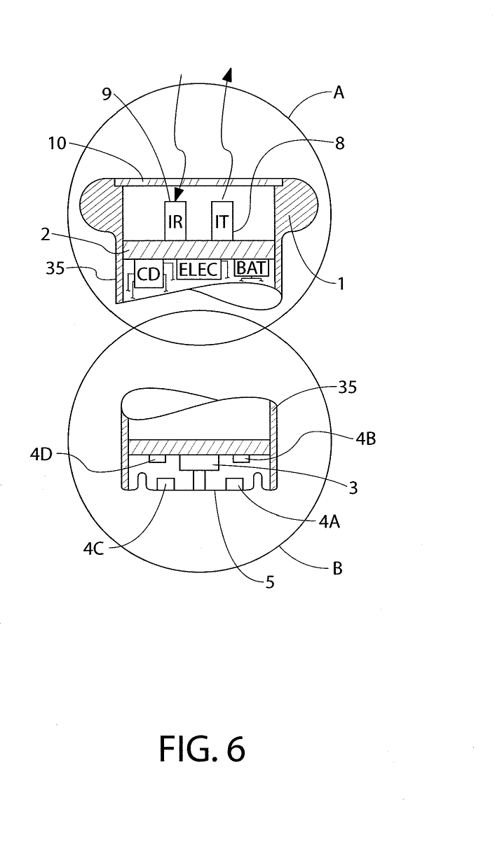

A further embodiment 120B, as shown in FIGS. 5-6, distributes the implantable sensor at the proximal and distal ends of a catheter 35. In particular, as shown most clearly in FIG. 5, the communication portion A of the sensor 120A is positioned at the proximal end of the catheter 35 which is located within the subarachnoid space 11; the pressure sensing portion B is located at the distal end of the catheter 35 within the brain ventricle 23 (FIG. 4). This configuration permits the pressure sensing portion B to be located within smaller and more critical areas of the brain without having to introduce the entire implantable pressure sensor 120A within such critical areas. It should be understood that the brain ventricle and subarachnoid space are shown by way of example only and that other implantation locations are within the broadest scope of the invention; the key feature is that the communication portion A is located more closely to the outside of the living being to facilitate the wireless communication with the remotely-located transceiver 122/122A while permitting the pressure sensing to occur within a deeper location within the living being.

### Implantable Sensor Calibration

The present invention solves some of the problems usually associated with implantable sensors. It provides with an easy calibration method which lessens stability requirements and enables obtaining the correct measured value (e.g. ICP), even if sensor offset or sensor sensitivity is altered. The key is that the sensor can be calibrated in situ once implanted.

### Calibrating for Membrane Stiffening

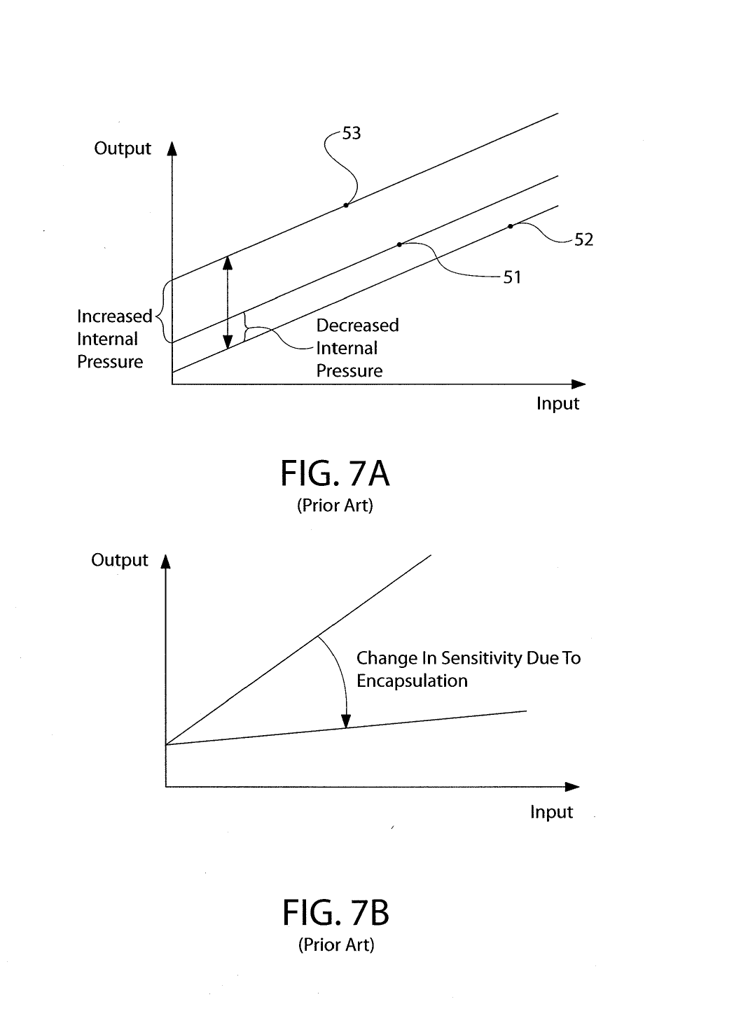

Once the sensor 120/120A is implanted within the living being, over time the membrane 5 is subjected to protein growths, among other things, and other factors that may cause the membrane to have a “stiffening” effect. As a result, there needs to be a way to account for that. To that end, the present invention 120-120A (FIGS. 1-6), includes the use of the capacitor actuator. The capacitor actuator comprises at least one capacitor 4A/4B and/or 4C/4D (e.g., modified capacitors—one or more) having one plate (e.g., 4A or 4C) mounted on the membrane 5 and the other plate (e.g., 4B or 4D, respectively) mounted internally, e.g., to the electronics board 2 of the sensor. The two plates (also referred to as “electrodes”) can move with respect to each other. They are not mechanically attached to each other. Charging each capacitor generates a force that pushes the respective capacitor's electrodes away from each other. This force pushes (or pulls) the membrane 5 with a well-calibrated force, thus the output of the force transducer 3 can be associated with a known force. Different calibrating forces can be applied, thus the current input-output characteristic of the sensor can be reconstructed (as depicted in FIG. 8A); by way of example only, the input-output characteristic (plot 40) can be obtained by application of three levels of force. For each force generated by the capacitor actuator (F¹_{C}, F²_{C} or F³_{C}) the output is O1, O2 or O3 is read. Those points can be then used to obtain a linear function: Output=A*F+offset, where A is constant. This can be subsequently converted to an ICP-output characteristic by supplementing F with ICP*S where S is the surface area of the membrane (see FIG. 8B). This process should be repeated rapidly so internal sensor housing pressure and ICP do not change between F¹_{C}, F²_{C} and F³_{C} measurements.

Thus, using capacitive actuators, multipoint calibration can be performed. The charge corresponding to certain force is applied F¹_{C}, F²_{C} and F³_{C}, and the output of the force transducer is measured. This process is repeated two or more times giving a series of input-output values corresponding to different forces generated by the capacitive actuators. This allows one to build a force output characteristic (see FIG. 8A) and then a corresponding ICP-output characteristic (see FIG. 8B). The calibration procedure can be repeated multiple times during implantation.

### Force Transducer Calibration

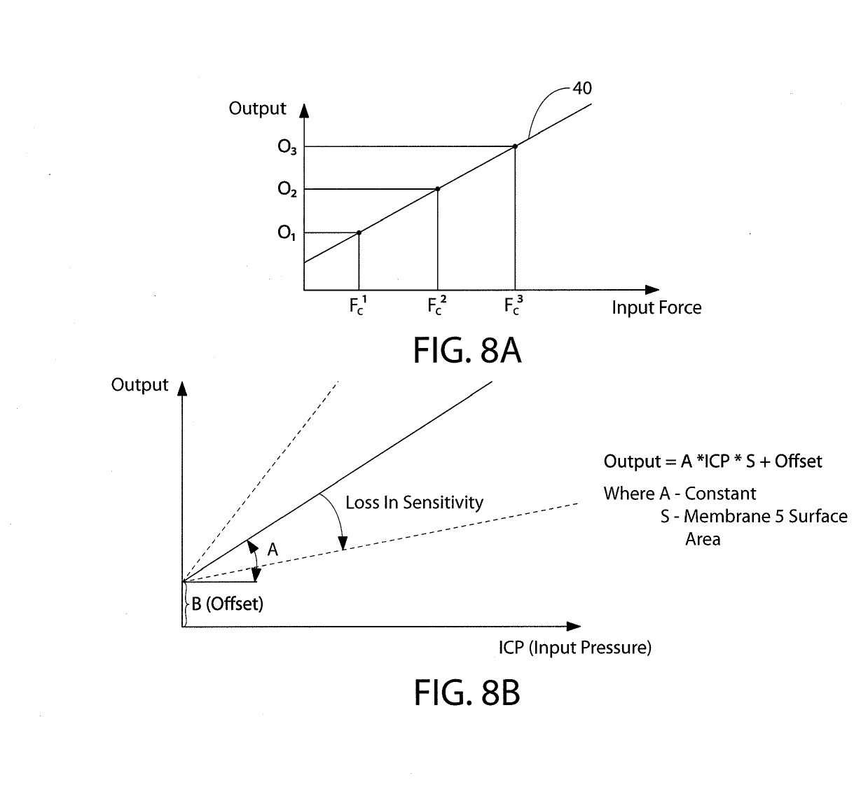

Every sensor carries an inherent risk of drifting with time. While several compensation methods exist for external sensors, the drift problem is accentuated in the case of an implantable sensor. The active element of the sensor (e.g., piezoresistive element or die) changes its properties with time, temperature etc. FIG. 9 depicts the variance of output vs. measured quantity (e.g., pressure) as temperature changes. The lower line 9A in FIG. 9 represents the normal operation curve of the die when operating at a temperature T₁. The slope of this line 9A represents the sensitivity of the sensor at that temperature. If the temperature is increased, the piezoresistive die's response to changes in pressure also changes (see upper line 9B in FIG. 9); in particular, the sensitivity changes and also an offset component is introduced. Such factors can be resolved by hardware and, typically, sensor housings are constructed with built-in compensation. However, such solutions increase the size of the sensor and the power consumption.

Moreover, changes in temperature produce changes in the pressure inside the sensor housing 120/120A. As shown most clearly in FIG. 9A, the force transducer 3 is a silicone die that has a very thin sensitive membrane 110 that is connected to the pressure on the outer side and to a diaphragm 111 in the inside. When the sensor housing 120/120A is filled with air, a rise in temperature generates an associated rise in the internal pressure. Such a pressure is directly outwardly, in opposition to the outside pressure (e.g., ICP) which would normally force the membrane 5 toward the interior of the sensor housing; thus, the detected value does not reflect the actual pressure.

Another source of drift might be related to sensor aging. However, the use of solid state components assures the longevity of the materials.

A typical solution to these problems is to utilize two identical sensors which respond to temperature and aging the same way. One sensor is usually exposed to the measured quantity while the reference one is only exposed to conditions inside the sensor housing. The resulting signal is calculated as a difference between the reference signal and the second sensor. However, this solution has several drawbacks: e.g., the reference pressure in the reference transducer has to be kept constant.

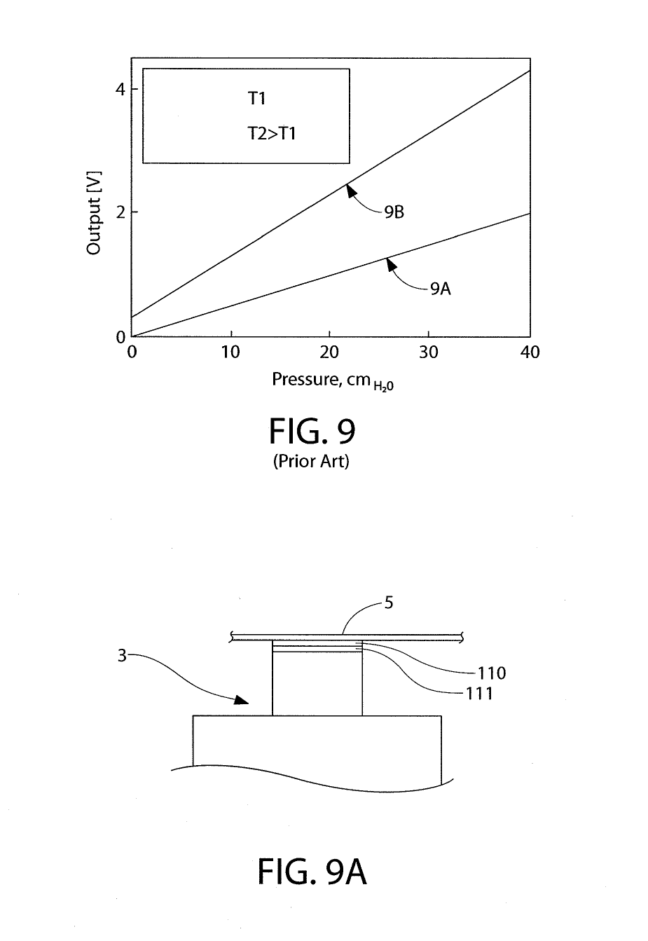

To address this concern, the present invention involves the following calibration technique on the force transducer. In particular, the method involves calibrating the sensor in-place before the measured quantity (e.g., ICP) reading is taken. This calibration technique assures that the parameters that affect the reading are taken into account and therefore their effects are nullified. The calibration method comprises four steps, as shown in FIG. 10:

Step I involves having the force transducer 3 in contact with the membrane 5. Step II involves displacing the force transducer 3 away from membrane 5 so that it is out of contact with the membrane 5 and a force transducer output is taken; this is the “zero pressure force” measurement. Step III involves applying a calibration force (e.g., a known constant amplitude force; the force transducer measures each calibration force and then the corrected characteristic is calculated by the accompanying electronics ELEC 7) to the force transducer and then taking a reading; this is the “calibration force” measurement. From these two points, a force transducer characteristic can be generated for this particular force transducer. With the force transducer characteristic generated, Step IV is initiated which returns the force transducer into contact with the membrane 5, where the measured quantity (e.g., ICP) reading is taken.

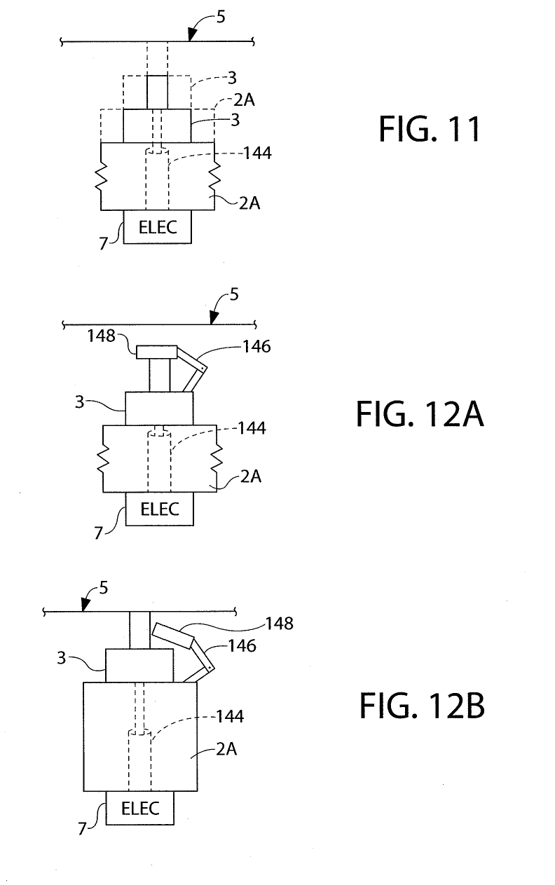

The calibration force can be accomplished using any well-known mechanisms 148 (see FIGS. 12A-12B) such as, but not limited to:
Actuator (e.g. piezoelectric cantilever)
Weight
Surface tension of the liquid (capillary tension)
Electrostatic charge
Magnet
Elastic elements (spring, cantilevers)
Or combinations of all above

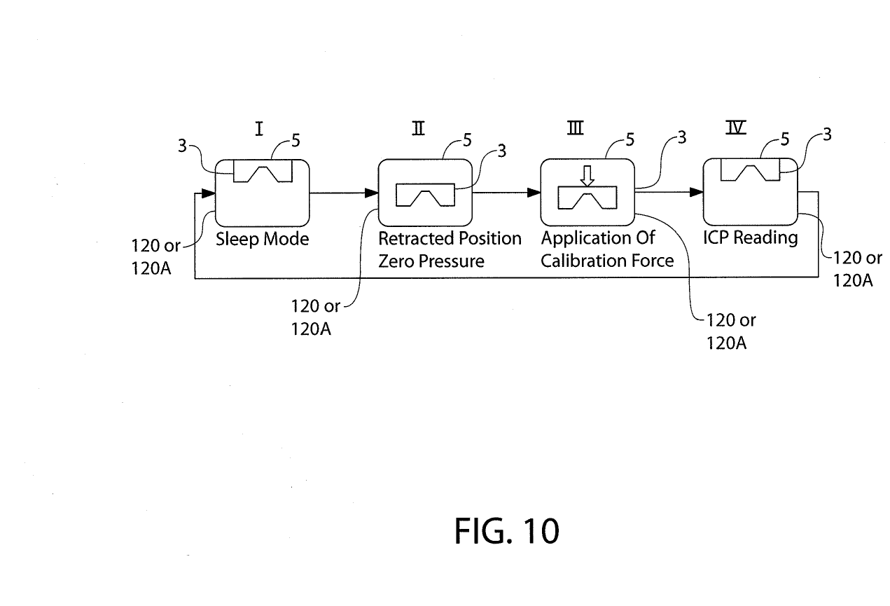

FIG. 11 shows the force transducer in its displaced condition, out of contact with the membrane 5, and in its operative condition (shown in phantom) with the force transducer in contact with the membrane 5. The force transducer 3 is fixedly secured to a portion 2A of the electronic board 2. Portion 2A is expandable to allow the force transducer 3 to be displaced. An actuator (e.g., telescoping actuator) 144 internal to the electronic board 2 displaces the force transducer 3 as commanded by the microcontroller 123. This actuator 144 causes the portion 2A to expand or contract vertically to displace the force transducer 3 either into contact with the membrane (operative condition) or out of contact (calibrating condition) with the membrane, respectively.

FIGS. 12A-12B depict how a calibrating force mechanism is positioned with respect to the force transducer depending on its operative or calibrating condition. A calibrating force member (as discussed above) 148 is disposed at one end of a bell crank 146 structure that is pivotable. As shown in FIG. 12A, when the actuator 144 displaces the force transducer 3 away from the membrane 3, in accordance with Step II, the bell crank 146 pivots, thereby positioning the calibrating member closely adjacent the force transducer 3. In this position, the calibrating member is not initially energized (by the microcontroller 123) in order for the zero pressure force measurement to be taken; once the zero pressure force measurement is taken, the calibrating member is energized to provide the calibrating force, as described above in Step III. FIG. 12B shows that, once the force transducer characteristic is generated, the actuator 144 displaces the force transducer 3 into its operative condition which rotates the bell crank 146, thereby moving the calibrating member 148 away from the force transducer 3 which then comes to rest against the membrane 3, in accordance with Step IV.

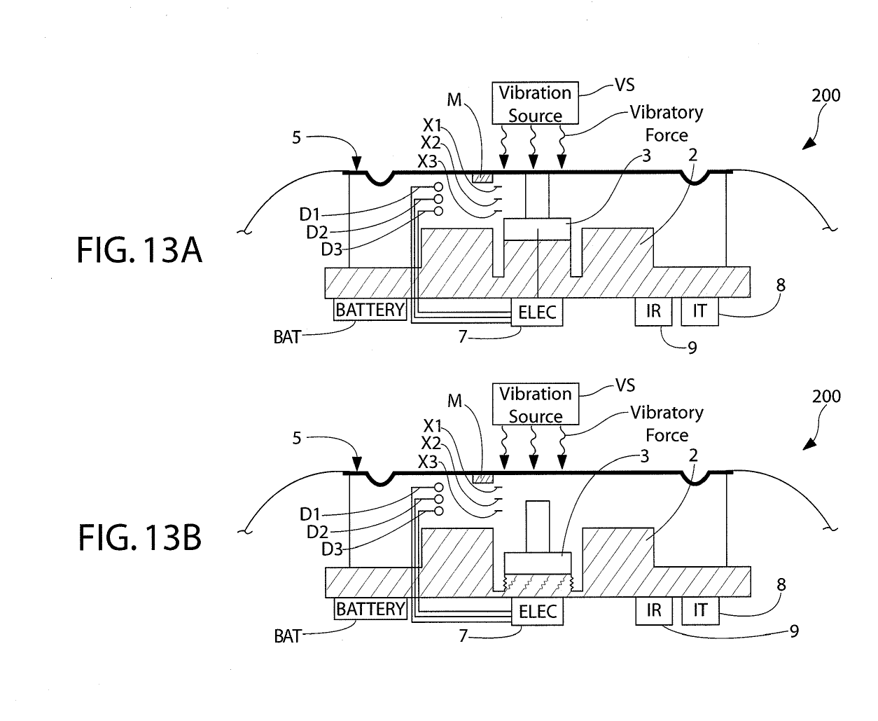

FIGS. 13A-13B depict an alternative configuration 200 of the implantable pressure sensor that does not utilize capacitive actuators but rather uses a dynamic method of recalibration. In this alternative method, the device 200 is vibrated by an external device, e.g., a vibratory source VS. The transducer sensing area (e.g., the membrane 5) has a known mass M coupled thereto. The mass M does not influence a slow signal (i.e., static case) transduction, such as intracranial pressure, but with rapid changes it produces a measurable force acting on the sensing area of the membrane 5. The displacement of the sensing area is monitored by a miniature optical device may comprise multiple pairs of photodiodes (e.g., transmitter-receiver pairs) or single diode detectors D1-D3 (by way of example only), etc. The multiple pairs of photodiodes or detectors D1-D3 detect when the sensing area of the membrane 5 reaches positions x1, x2 and x3 and send a signal to the onboard microcontroller 123 to register the time to travel between x1, x2 and x3. The calibrated force is calculated as F=m*d²x/de, where x is the distance. The advantages of this method are:
1) it is based on distance and time measurements which are independent of internal pressure and temperature; and
2) it uses mostly external power to generate the force acting on the transducer (i.e., the force is generated by inertia of the vibrating mass M and an externally generated acceleration, a).

As shown in FIG. 13A, with the force transducer 3 in contact with the membrane 5, the overall sensor 100 or 100A may be calibrated. In addition, as shown in FIG. 13B, with the force transducer 3 displaced away from the membrane 5 (using the displacement actuator discussed previously), the membrane 5 may be calibrated.

While the invention has been described in detail and with reference to specific examples thereof, it will be apparent to one skilled in the art that various changes and modifications can be made therein without departing from the spirit and scope thereof.

1. A pressure sensor that is implantable within a living being for detecting a pressure present at a location wherein said pressure sensor is implanted, said implantable pressure sensor comprising:
a housing comprising one side formed by a flexible membrane;
said housing further comprising sensor electronics including a force transducer in contact with said membrane for detecting flexing of said flexible membrane when said flexible membrane is exposed to the pressure present at the location;
said sensor electronics further comprising at least one capacitor coupled to said flexible membrane, said at least one capacitor applying a known force to said membrane, detected by said force transducer, when said at least one capacitor is energized by said sensor electronics; and
wherein said known force is used to calibrate for a stiffness associated with said flexible membrane in measuring the pressure at the location.

2. The pressure sensor of claim 1 wherein said at least one capacitor comprises a pair of capacitor plates wherein a first capacitor plate is secured to said flexible membrane and a second capacitor plate is fixed within said housing and aligned with said first plate.

3. The pressure sensor of claim 2 further comprising a second capacitor comprising a second pair of capacitor plates that are arranged similarly to said first and second capacitor plates.

4. The pressure sensor of claim 1 further comprising a radio frequency transmitter for transmitting the measured pressure at the location to a remotely-located receiver.

5. The pressure sensor of claim 1 further comprising an infrared transmitter for transmitting the measured pressure at the location to a remotely-located receiver.

6. The pressure sensor of claim 5 further comprising an infrared receiver for receiving a start command from a remotely-located transmitter.

7. The pressure sensor of claim 6 further comprising a rechargeable battery and wherein said rechargeable battery obtains its recharging energy via said infrared receiver.

8. The pressure sensor of claim 5 wherein said housing comprises a transparent surface, said infrared transmitter being located within said housing adjacent said transparent surface.

9. The pressure sensor of claim 1 wherein said force transducer is displaceable within said housing.

10. The pressure sensor of claim 1 wherein said sensor electronics further comprises a calibrating force member, said calibrating force member applying a known calibrating force to said force transducer when said force transducer is displaced away from said membrane.

11. The pressure sensor of claim 1 wherein said location wherein said pressure sensor is implanted is the head of the living being and wherein said pressure present at a location is intracranial pressure (ICP).

12. The pressure transducer of claim 6 further comprising a catheter having a proximal end and a distal end, said distal end comprising said force transducer, said membrane and said at least one capacitor disposed at a first location within the living being and wherein said proximal end comprises said infrared transmitter and infrared receiver disposed at a second location within the living being, said second location between closer to an outside surface of the living being than said first location.

13. The pressure sensor of claim 12 wherein said first location comprises the brain ventricle of the living being and the second location comprises the subarachnoid space and wherein said pressure present at a location is intracranial pressure (ICP).

14. A pressure sensor that is implantable within a living being for detecting a pressure present at a location wherein said pressure sensor is implanted, said implantable pressure sensor comprising:
a housing comprising one side formed by a flexible membrane;
said housing further comprising sensor electronics including a displaceable force transducer in contact with said membrane for detecting flexing of said flexible membrane when said flexible membrane is exposed to the pressure present at the location;
said sensor electronics further comprising a calibrating force member that applies a known calibrating force to said force transducer when said force transducer is displaced away from said flexible membrane; and
wherein said known force is used, along with a zero pressure value obtained when said force transducer is displaced away from said membrane and without application of said known calibrating force, to form a force transducer characteristic which regulates all future force transducer measurements.

15. The pressure sensor of claim 14 wherein said sensor electronics further comprise at least one capacitor applying a known force to said membrane, detected by said force transducer, when said at least one capacitor is energized by said sensor electronics and wherein said known force is used to calibrate for a stiffness associated with said flexible membrane in measuring the pressure at the location.

16. The pressure sensor of claim 14 further comprising a radio frequency transmitter for transmitting the measured pressure at the location to a remotely-located receiver.

17. The pressure sensor of claim 14 further comprising an infrared transmitter for transmitting the measured pressure at the location to a remotely-located receiver.

18. The pressure sensor of claim 17 further comprising an infrared receiver for receiving a start command from a remotely-located transmitter.

19. The pressure sensor of claim 18 further comprising a rechargeable battery and wherein said rechargeable battery obtains its recharging energy via said infrared receiver.

20. The pressure sensor of claim 17 wherein said housing comprises a transparent surface, said infrared transmitter being located within said housing adjacent said transparent surface.

21. The pressure sensor of claim 14 wherein said location wherein said pressure sensor is implanted is the head of the living being and wherein said pressure present at a location is intracranial pressure (ICP).

22. The pressure transducer of claim 18 further comprising a catheter having a proximal end and a distal end, said distal end comprising said force transducer, said membrane and said at least one capacitor disposed at a first location within the living being and wherein said proximal end comprises said infrared transmitter and infrared receiver disposed at a second location within the living being, said second location between closer to an outside surface of the living being than said first location.

23. The pressure sensor of claim 22 wherein said first location comprises the brain ventricle of the living being and the second location comprises the subarachnoid space and wherein said pressure present at a location is intracranial pressure (ICP).

24. A method for calibrating a pressure sensor in situ within a living being for detecting a pressure present at a location within the living being, said method comprising:
disposing a pressure sensor within the living being wherein the pressure sensor comprises a force transducer in contact with a flexible membrane, forming a portion of an outer surface of said pressure sensor, that is exposed to the pressure present at the location;
coupling a capacitor to said flexible membrane;
energizing said capacitor with a plurality of energy levels to apply corresponding known forces to said flexible membrane; and
collecting the force transducer outputs corresponding to said applied known forces to generate a flexible membrane characteristic that is used to account for membrane stiffness which regulates all future force transducer measurements.

25. The method of claim 24 further comprising calibrating said force transducer, said calibrating said force transducer comprising:
displacing said force transducer away from said flexible membrane;
collecting a force transducer output with said force transducer displaced out of contact with said flexible membrane to obtain a zero pressure value;
applying at least one known calibrating force to said force transducer and collecting a corresponding force transducer output; and
generating a force transducer characteristic from said zero pressure value and said corresponding force transducer output which further regulates all future force transducer measurements.

26. The method of claim 25 wherein said step of applying at least one known calibrating force comprises disposing a calibrating force member in close proximity to said force transducer.

27. The method of claim 24 wherein said step of coupling a capacitor to said flexible membrane comprises securing a first capacitor plate to said flexible membrane and securing a second capacitor plate, aligned with said first capacitor plate, within a sensor housing.

28. The method of claim 24 further comprising the step of wirelessly transmitting a force transducer output to a remotely-located receiver.

29. The method of claim 28 wherein said step of wirelessly transmitting a force transducer output is accomplished via a radio transmission.

30. The method of claim 28 wherein said of wirelessly transmitting a force transducer output is accomplished via an infrared transmission.

31. The method of claim 30 further comprising the step of recharging a battery within a sensor housing said infrared transmission.

32. The method of claim 24 wherein said step of disposing a pressure sensor within the living being comprises positioning said pressure sensor within the subarachnoid space of a living being to measure intracranial pressure (ICP).

33. The method of claim 24 wherein said step of disposing a pressure sensor within the living being comprises:
locating said force transducer, said flexible membrane and said at least one capacitor at a distal end of a catheter;
locating an infrared transmitter and an infrared receiver at a proximal end of said catheter;
positioning said catheter within the living being such that said distal end is located at a first location within the living being and said proximal end is at a second location within the living being, said second location being closer to an outside surface of the living being than said first location.

34. The method of claim 33 wherein said first location comprises the brain ventricle of the living being and the second location comprises the subarachnoid space and wherein said pressure present at a location is intracranial pressure (ICP).

35. A method for calibrating a pressure sensor in situ within a living being for detecting a pressure present at a location within the living being, said method comprising:
disposing a pressure sensor within the living being wherein the pressure sensor comprises a force transducer in contact with a flexible membrane, forming a portion of an outer surface of said pressure sensor, that is exposed to the pressure present at the location;
displacing said force transducer away from said flexible membrane;
collecting a force transducer output with said force transducer displaced out of contact with said flexible membrane to obtain a zero pressure value;
applying at least one known calibrating force to said force transducer and collecting a corresponding force transducer output; and
generating a force transducer characteristic from said zero pressure value and said corresponding force transducer output which regulates all future force transducer measurements.

36. The method of claim 35 further comprising calibrating said sensor with respect to membrane stiffness, said calibrating said sensor with respect to membrane stiffness comprising:
coupling a capacitor to the flexible membrane;
energizing said capacitor with a plurality of energy levels to apply corresponding known forces to said flexible membrane; and
collecting the force transducer outputs corresponding to said applied known forces to generate a flexible membrane characteristic that is used to account for membrane stiffness which further regulates all future force transducer measurements.

37. The method of claim 35 wherein said step of applying at least one known calibrating force comprises disposing a calibrating force member in close proximity to said force transducer.

38. The method of claim 36 wherein said step of coupling a capacitor to said flexible membrane comprises securing a first capacitor plate to said flexible membrane and securing a second capacitor plate, aligned with said first capacitor plate, within a sensor housing.

39. The method of claim 35 further comprising the step of wirelessly transmitting a force transducer output to a remotely-located receiver.

40. The method of claim 39 wherein said step of wirelessly transmitting a force transducer output is accomplished via a radio transmission.

41. The method of claim 39 wherein said of wirelessly transmitting a force transducer output is accomplished via an infrared transmission.

42. The method of claim 41 further comprising the step of recharging a battery within a sensor housing said infrared transmission.

43. The method of claim 35 wherein said step of disposing a pressure sensor within the living being comprises positioning said pressure sensor within the subarachnoid space of a living being to measure intracranial pressure (ICP).

44. The method of claim 35 wherein said step of disposing a pressure sensor within the living being comprises:
locating said force transducer, said flexible membrane and said at least one capacitor at a distal end of a catheter;
locating an infrared transmitter and an infrared receiver at a proximal end of said catheter;
positioning said catheter within the living being such that said distal end is located at a first location within the living being and said proximal end is at a second location within the living being, said second location being closer to an outside surface of the living being than said first location.

45. The method of claim 44 wherein said first location comprises the brain ventricle of the living being and the second location comprises the subarachnoid space and wherein said pressure present at a location is intracranial pressure (ICP).

46. A pressure sensor that is implantable within a living being for detecting a pressure present at a location wherein said pressure sensor is implanted, said implantable pressure sensor comprising:
a housing comprising one side formed by a flexible membrane;
said housing further comprising sensor electronics including a displaceable force transducer in contact with said membrane for detecting flexing of said flexible membrane when said flexible membrane is exposed to the pressure present at the location, said flexible member comprising a known mass coupled thereto;
said sensor electronics further comprising a processor coupled to at least one detector for detecting the displacement of said mass when a known vibratory force is applied to said flexible membrane; and
wherein said processor calculates a calibration force based on said displacement of said mass and time of displacement of said mass to form a force transducer characteristic which regulates all future force transducer measurements.

47. The pressure sensor of claim 46 wherein said processor calculates said calibration force with said force transducer out of contact with said flexible membrane.

48. A method for calibrating a pressure sensor in situ within a living being for detecting a pressure present at a location within the living being, said method comprising:
disposing a pressure sensor within the living being wherein the pressure sensor comprises a force transducer in contact with a flexible membrane, forming a portion of an outer surface of said pressure sensor, that is exposed to the pressure present at the location and wherein a known mass is coupled to said flexible membrane;
applying a known vibratory force to said flexible membrane and collecting displacement data of said known mass; and
generating a force transducer characteristic from said displacement data which regulates all future force transducer measurements.

49. The method of claim 48 wherein said force transducer is displaced away from flexible membrane when said known vibratory force is applied to said flexible membrane and said displacement data is collected.
